(19) United States
(12) Patent Application Publication
(10) Pub. No.: US 2004/0143220 A1
(43) Pub. Date: Jul. 22, 2004

(54) Wound and catheter dressing and a method for making and applying a dressing

(76) Inventor: George Worthley, Wheaton, IL (US)

Correspondence Address: PATENTS+TMS, A Professional Corporation, 1914 North Milwaukee Avenue, Chicago, IL, 60647, US

(21) Appl. No.: 10/346,893

(22) Filed: Jan. 17, 2003

(51) Int. Cl. A61M 5/32

(52) U.S. Cl. 604/174; 128/DIG. 6; 428/343

(57) ABSTRACT

A film dressing for intravenous catheter sites as well as a method for making and applying such a dressing are provided. Preferably, the film dressing has a film layer attached to a second layer, a third layer, at least two adhesive layers and a silicone coated liner supporting the dressing. The second layer may be constructed of a non-woven material. The third layer may be either a polyurethane film layer or a polyethylene layer. Additional strips may be used to secure the dressing on a patient and/or to secure a catheter. The dressing may have a notch for the catheter. A label may be removably attached to the silicone coated liner.

### BACKGROUND OF THE INVENTION

The present invention generally relates to a film dressing for intravenous catheter sites as well as a method for making and applying such a dressing. More specifically, the present invention relates to a dressing having three layers, a plurality of adhesive layers, and a a liner for protection of the dressing. The dressing of the present invention further has a notch for accommodating a catheter and a window for viewing of the catheter site.

It is, of course, generally known to use film dressings for covering of wound and/or intravenous catheter sites. Dressings that are self-adherent are also known. Such dressings generally have a transparent polyurethane film with an adhesive and a liner. Other known dressings have a transparent film with an adhesive and a liner releasably adhered to the exposed adhesive on the film. Further, other known dressings have a window for viewing of the wound and/or catheter insertion area. Known dressings generally have a cut in the dressing for receiving a catheter tube.

Further, many medical procedures involve the use of a catheter inserted into the body of a patient for fluid infusion or removal. The catheter often remains secured to the patient for many days. During this period, the catheter is positioned and is accessible for attachment and detachment of fluid handling devices. A dressing to anchor or position the catheter and allow access to the catheter insertion site is known. The known dressing allows for visually locating the catheter insertion site and protecting the site; however, one problem associated with this dressing includes comfortably securing and/or anchoring a catheter and/or tubing to the patient. Further, it is a common practice to apply strips of adhesive tape across a catheter at a placement site to secure and protect the placement. Tape may retain the catheter in position, but is not flexible and may be uncomfortable for the patient.

A need, therefore, exists for a dressing for an intravenous catheter site and a method for making and a method for applying such a dressing that is easy to use, comfortable for the patient, securely seals the catheter to the patient and/or dressing and allows a practitioner to examine the catheter insertion site. Further, a need exists for a dressing that is inexpensive to manufacture.

### SUMMARY OF THE INVENTION

The present invention generally relates to a film dressing to cover a catheter site and a method for applying and making such a dressing. Preferably, the film dressing has three layers of film and/or a non-woven material, a notch for receiving a catheter, and a window for viewing the catheter insertion site. The dressing may further have strips for use in the application of the dressing to a patient. Further, the dressing may have a liner coated with silicone for easy removal of the liner from the dressing and a label on the dressing to provide information relating to the patient.

To this end, in an embodiment of the present invention, a dressing is provided having a first film, a first layer, a first adhesive, a second layer and a liner. The first film has a right edge, a left edge, a top side and a bottom side. The first layer has a top surface and a bottom surface wherein the top surface of the first layer is attached to the bottom side of the first film. The first adhesive is on the bottom surface of the first layer. The second layer has a top face, a bottom face and a second adhesive on the bottom face wherein the top face of the second layer is attached to the bottom surface of the first layer by the first adhesive. The liner has a first side and a second side and a non-adhesive coating on the first side wherein the liner is removably attached to the bottom face of the second layer.

In an embodiment, the dressing has a notch in the first film.

In an embodiment, the dressing has a strip attached to the top side of the first film.

In an embodiment, the dressing has a window in the second layer.

In an embodiment, the dressing has a hinge secured to the second layer.

In an embodiment, the dressing has a third adhesive securing the first layer to the bottom side of the first film.

In an embodiment, the dressing has a second non-adhesive layer on the second side of the liner.

In an embodiment, the dressing has a second liner having a top and a bottom wherein the bottom of the second liner is secured to the top side of the first film.

In an embodiment, the dressing has a second liner having a strip wherein the second liner is secured to the top side of the first film.

In an embodiment, the dressing has a label removably attached to the second side of the liner.

In an embodiment, the first layer of the dressing is made from a foam.

In an embodiment, the first layer of the dressing is made from a film.

In an embodiment, the first film is made from a polyethylene material.

In an embodiment, the first film is made from a polyurethane material.

In an embodiment, the second layer is made from a foam.

In an embodiment, the first layer is made from a non-woven polyester material.

In an embodiment, the first adhesive includes acrylic.

In another embodiment of the present invention, a dressing is provided having a frame, a first film, a second adhesive, a second film, and a third adhesive. The frame has a top and a bottom wherein the bottom of the frame has an adhesive. The first film has a top side and a bottom side wherein the top side of the first film is attached to the bottom of the frame. The second adhesive is on the bottom side of the first film and the third adhesive is on a bottom surface of the second film. The second film has a top surface and a bottom surface wherein the top surface of the second film is attached to the bottom side of the first film by the second adhesive.

In an embodiment, the dressing has a second liner secured to the bottom surface of the second film by the third adhesive.

In an embodiment, the dressing has a non-adhesive layer on the second liner.

In an embodiment, the dressing has a third film attached to the bottom surface of the second film by the third adhesive.

In an embodiment, the frame of the dressing has a shape and the first film has a shape wherein the shape of the first film is substantially the same as the shape of the frame.

In another embodiment of the present invention, a method for applying a dressing to a catheter insertion site on a patient is provided. The method comprises the steps of: providing a frame having a top and a bottom wherein the bottom of the frame has an adhesive; providing a first film having a bottom side; attaching the first film to the bottom of the frame with the adhesive; providing a first layer having a top surface and a bottom surface; attaching the top surface of the first layer to the bottom side of the first film; providing a second layer having a top face and a bottom face wherein the top face of the second layer is on the bottom surface of the first layer; and exposing the bottom face of the second layer.

In an embodiment, the method for applying a dressing to a catheter insertion site on a patient further comprises the step of placing the second layer on the patient.

In an embodiment, the method for applying a dressing to a catheter insertion site on a patient further comprises the step of securing the dressing on the patient with an adhesive strip.

In another embodiment of the present invention, a method for manufacture of a dressing is provided. The method comprises the steps of: providing a temporary liner with an adhesive; attaching a first film to the temporary liner with the adhesive; removing the temporary liner; attaching a first layer to the first film; providing a second layer having a top face, a bottom face and having a second adhesive on the bottom face; attaching the first film to the top face of the second layer with the adhesive; securing a liner to the second layer; and attaching a second liner to the first layer.

In an embodiment, the method for manufacture of a dressing further comprises the step of curing the adhesive on the temporary liner.

In an embodiment, the method for manufacture of a dressing further comprises the step of forming a notch in the dressing.

In an embodiment, the method for manufacture of a dressing further comprises the step of attaching the second liner to the bottom face of the second layer with the second adhesive.

In an embodiment, the method for manufacture of a dressing further comprises the step of attaching a removable label to the liner.

In an embodiment, the first layer is a polyurethane film.

In an embodiment, the first film is a polyethylene film.

In an embodiment, the second layer is a polyethylene foam.

In an embodiment, the method for manufacture of a dressing further comprises the step of securing a strip to the liner.

In an embodiment, the method for manufacture of a dressing further comprises the step of forming a window in the second layer.

In an embodiment, the adhesive includes acrylic.

It is, therefore, an advantage of the present invention to provide a dressing and a method for making and applying such a dressing to cover a catheter site.

Another advantage of the present invention is to provide a dressing and a method for making and applying such a dressing wherein the dressing is easy to use and inexpensive to manufacture.

Another advantage of the present invention is to provide a dressing and a method for making and applying such a dressing wherein the dressing allows access for attachment and detachment of handling devices.

Further, an advantage of the present invention is to provide a dressing and a method for making and applying such a dressing wherein the dressing is securely adhered to a patient and/or catheter.

A still further advantage of the present invention is to provide a dressing and a method for making and applying such a dressing wherein the dressing has a non-woven film layer to conform to catheter tubing.

Another advantage of the present invention is to provide a dressing and a method for making and applying such a dressing wherein the dressing has a flexible layer for patient comfort.

Yet another advantage of the present invention is to provide a dressing and a method for making and applying such a dressing wherein a label on the dressing provides patient identification and/or information.

Additional features and advantages of the present invention are described in, and will be apparent from, the detailed description of the presently preferred embodiments and from the drawings.

### BRIEF DESCRIPTION OF THE DRAWINGS

FIG. 1 is a top view of a dressing in an embodiment of the present invention.

FIG. 2 is a cross-sectional view of a dressing taken generally along the line II-II of FIG. 1 in an embodiment of the present invention.

FIG. 3 is a top view of a dressing in another embodiment of the present invention.

FIG. 4 is a cross-sectional view of a dressing taken generally along the line IV-IV of FIG. 3 in an embodiment of the present invention.

FIG. 5 is a top view of a dressing in another embodiment of the present invention.

FIG. 6 is a cross-sectional view of a dressing taken generally along the line VI-VI of FIG. 5 in an embodiment of the present invention.

FIG. 7 is a top view of a dressing in another embodiment of the present invention.

FIG. 8 is a cross-sectional view of a dressing taken generally along the line VIII-VIII of FIG. 7 in an embodiment of the present invention.

FIG. 9 is a top view of a dressing in another embodiment of the present invention.

FIG. 10 is a cross-sectional view of a dressing taken generally along the line X-X of FIG. 9 in an embodiment of the present invention.

FIG. 11 is a top view of a dressing in another embodiment of the present invention.

FIG. 12 is a cross-sectional view of a dressing taken generally along the line XII-XII of FIG. 11 in an embodiment of the present invention.

FIG. 13 is a flowchart illustrating the steps for applying a dressing to a patient in an embodiment of the present invention.

FIG. 14 is a flowchart illustrating the steps for applying a dressing to a patient in another embodiment of the present invention.

FIG. 15 is a flowchart illustrating the steps for applying a dressing to a patient in another embodiment of the present invention.

FIG. 16 is a flowchart illustrating the steps for applying a dressing to a patient in another embodiment of the present invention.

FIG. 17 is a flowchart illustrating the steps for making a dressing in an embodiment of the present invention.

FIG. 18 is a flowchart illustrating the steps for making a dressing in another embodiment of the present invention.

FIG. 19 is a flowchart illustrating the steps for making a dressing in another embodiment of the present invention.

FIG. 20 is a flowchart illustrating the steps for making a dressing in another embodiment of the present invention.

### DETAILED DESCRIPTION OF THE PRESENTLY PREFERRED EMBODIMENTS

The present invention generally relates to a film dressing and a method for making and applying the same. The dressing may have a first layer or first film, a second film and an adhesive wherein the first layer or the first film and the second film are secured by the adhesive. The dressing may further have strips to secure the dressing and/or a catheter to a patient.

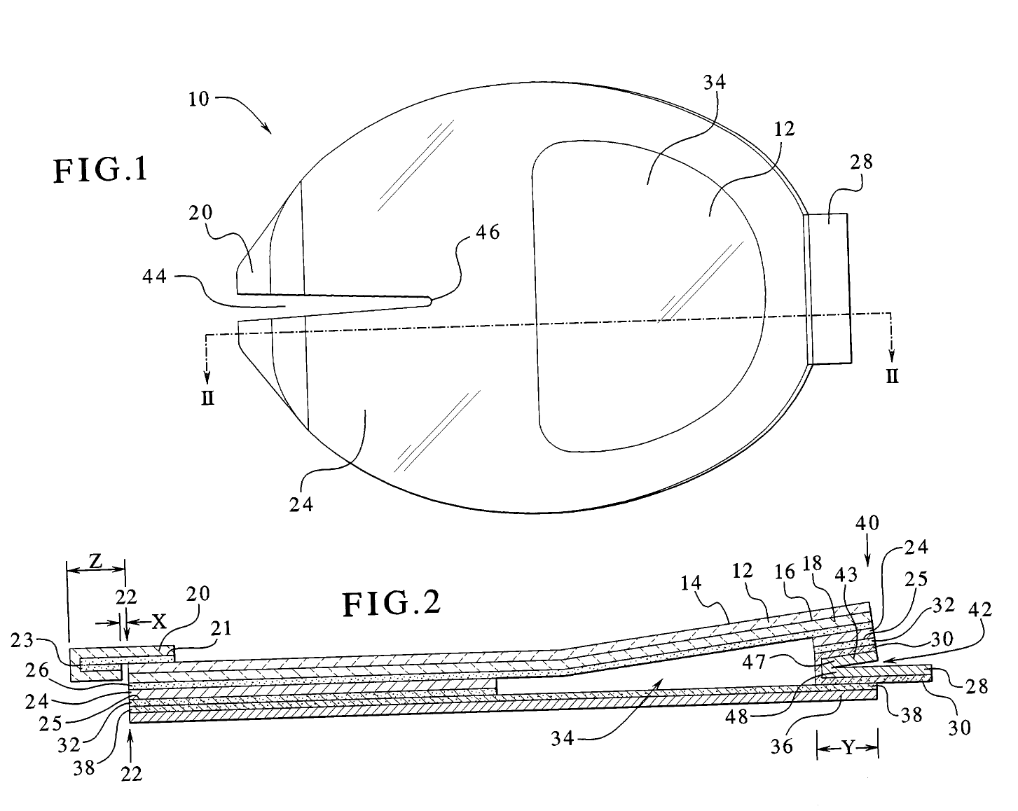

Referring now to the drawings wherein like numerals refer to like parts, FIGS. 1 and 2 illustrate a dressing 10 of the present invention. As illustrated in FIGS. 1 and 2, the dressing 10 may have a first film 12 with a top side 14 and a bottom side 16. Preferably, the first film 12 may be constructed from polyethylene and may be transparent. Further, the first film 12 may have a thickness between 0.5 and 3.0 millimeters, preferably 2.0 millimeters. A second film 18 may be attached to the first film 12 on the bottom side 16 of the first film 12. Preferably, the second film 18 may be constructed from polyurethane and may be transparent. Further, the second film 18 may have a thickness between 0.5 and 3.0 millimeters, preferably 1.0 millimeters. A gripping strip 20 may be attached to the first film 12. A bottom surface 21 of the gripping strip 20 may have an adhesive coating 23 that may adhere to the top side 14 of the first film 12 at a distance from the first edge 22 of the first film 12. The gripping strip 20 may extend a distance “Z” from the first edge 22 and may be folded so that the bottom surface 21 of the gripping strip 20 may attach to itself a distance “X” from the right edge 22. The gripping strip 20 may be formed, for example, from cellophane tape.

A layer 24 may be attached to the second film 18 by a first adhesive 26 such that the second film 18 is between the layer 24 and the first film 12. Preferably, the layer 24 is made from a non-woven material such as, for example, polyester. The non-woven material may be flexible and soft to the touch to provide additional comfort to the patient. The non-woven material preferably has a density of 1.3 ounces per square yard. Alternatively, the non-woven material may have a density of five ounces per square yard. Further, the first adhesive 26 preferably has a thickness between 1.0 and 2.0 millimeters. A first liner 28 having a first silicone coating 30 may be attached to the layer 24 by a second adhesive 32 on a bottom surface 25 of the layer 24. The dressing 10 may further have a window 34 in the layer 24 and in the second adhesive 32 as shown in FIG. 2. The layer 24 may be made from a material that is absorptive. Alternatively, the layer 24 may be made from a material that is non-absorptive.

A second liner 36 with a silicone coating 38 may be incorporated into the dressing 10. The second liner 36 may be attached to the layer 24 at the first edge 22 of the dressing 10 and extend towards the second edge 40. The first liner 28 may be removably attached to the layer 24 at a second edge 40 of the dressing 10 and may fold at a distance “Y” from the second edge 40 of the dressing 10. More specifically, the configuration of the first liner 28 may define a hinge 42.

As further shown in FIG. 2, the hinge 42 may have a top side 43 and a bottom side 44 and may be removably attached to the layer 24 and the second liner 36. The top side 43 of a first portion 47 of the hinge 42 may be removably attached to the layer 24 at a distance from the second edge 40 of the layer 24. The hinge 42 may be folded onto itself wherein the first portion 47 of the hinge 42 may be removably attached to the layer 24. A second portion 48 of the hinge 42 may extend between the first portion 47 of the hinge 42 and the second liner 36. The second portion 48 of the hinge 28 may extend at a distance beyond the second edge 40 of the dressing 10. The second portion 48 of the hinge 42 may extend a distance beyond the second edge 40 of the dressing 10. The second portion 48 may have printed information (not shown) on the hinge 42. Information on the hinge may include, for example, to remove the hinge 42 after removing the second liner 36 when applying the dressing 10.

Referring again to FIG. 1, the dressing 10 may have a notch 44 having an end 46 to accommodate a cylindrical tube, such as, for example, a catheter. Preferably, the end 46 is semi-circular in shape. The notch 44 may be circular in shape. Alternatively, the notch 44 may be a slit, or any other shape that may accommodate a catheter and/or tubing. The layer 24 and the second film 18 may be flexible thereby allowing the notch 44 to easily and securely accept a catheter.

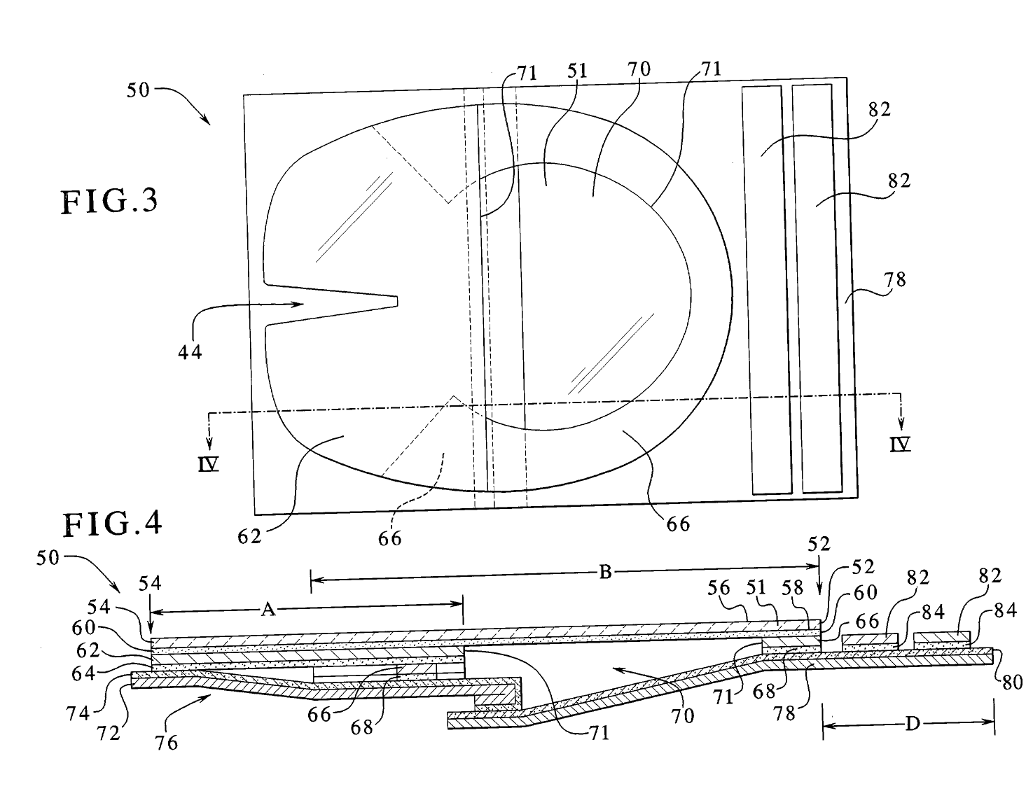

Referring now to FIGS. 3 and 4, in an embodiment of the present invention, a dressing 50 may have a first film 51 having a right edge 52, a left edge 54, a top 56, a bottom 58 and a first adhesive 60 on the bottom 58 of the first film 51. Preferably, the first film 51 may be constructed from polyurethane and may be transparent. Further, the first film 51 may have a thickness between 0.5 and three millimeters, preferably one millimeter. Preferably, the first adhesive 60 has a thickness between one and two millimeters. A layer 62 may be attached to the first adhesive 60 on the bottom 58 of the first film 51. The layer 62 may be constructed from a non-woven material. The non-woven material preferably has a density of 1.3 ounces per square yard. Alternatively, the non-woven material may have a density of five ounces per square yard. The layer 62 may extend from the left edge 54 to a distance “A” from the left edge 54.

A second adhesive 64 may be provided on the layer 62 on a side opposite to the first film 51. Preferably, the second adhesive 64 has a thickness between 1 and 2 millimeters. A foam 66 may be attached to the first adhesive 60 on the bottom 58 of the first film 51 and may be attached to the second adhesive 64. Preferably, the foam 66 may be constructed from polyethylene and may have a thickness of one-sixteenth of an inch. The foam 66 may extend from the right edge 52 to a distance “B” from the right edge 52. A third adhesive 68 may be provided on the foam 66 on a side opposite the first film 51. The third adhesive 68 may also be provided on the foam 66 on a side opposite the layer 62. Preferably, the third adhesive 68 has a thickness between one and two millimeters. A window 70 may be formed by cutting through the foam 66 and the third adhesive 68. Further, the foam 66 and the layer 62 may define a perimeter 71 around the window 70.

The dressing 50 may further have a first liner 72 and a second liner 78. The configuration of the first liner 72 may define a hinge 76. The second liner 78 may be attached to the third adhesive 68 and may extend from a distance “C” from the left edge 54 to a distance “D” from the right edge 52. Preferably, the first liner 72 may have a first silicone layer 74, and the second liner 78 may have a second silicone layer 80.

Further, the dressing 50 may have a strip 82 attached to the second liner 78 and next to the foam 66. Preferably, the strip 82 is constructed from a non-woven material such that the strip 82 is flexible. The strip 82 may be attached to the second liner 78 with an adhesive 84. The dressing 50 illustrated in FIG. 3 may also have a notch 44 as described above.

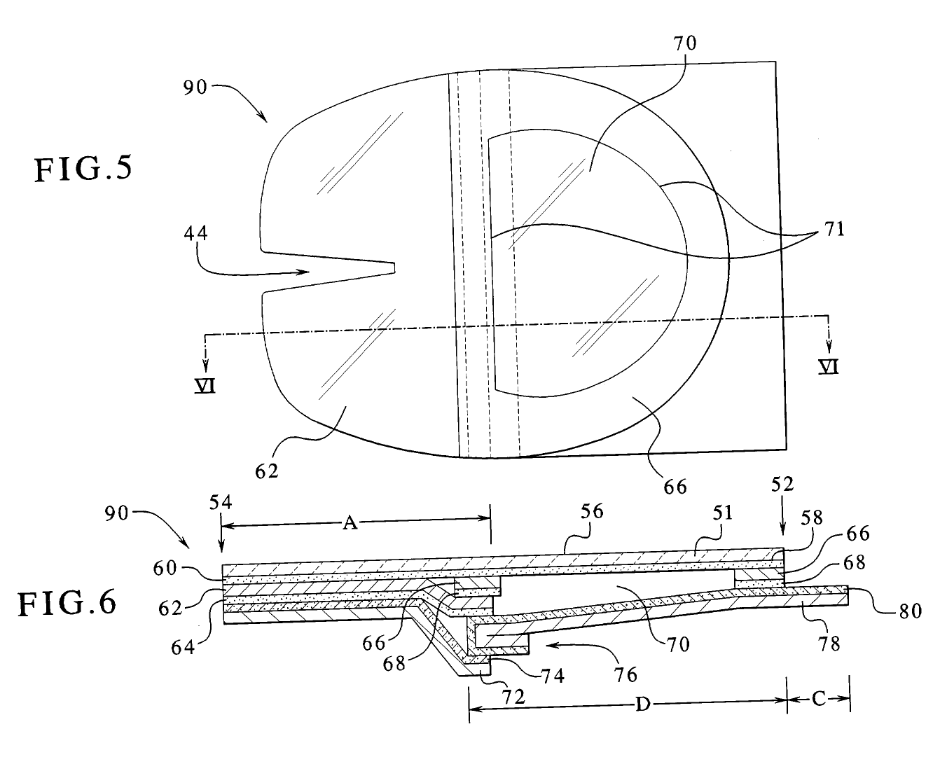

Referring now to FIGS. 5 and 6, in an embodiment of the present invention, a dressing 90 may have substantially the same configuration as the embodiment illustrated in FIGS. 3 and 4, except that the layer 62 may be attached to both the first adhesive 60 and the third adhesive 68. More specifically, the dressing 90 may have the layer 62 attached to the first film 51 by the first adhesive 60 and the layer 62 may also be attached to the foam 66 by the third adhesive 68.

Further, in the embodiment illustrated in FIGS. 5 and 6, the first liner 72 may be removably attached to the second adhesive 66. The second liner 78 may be attached to the third adhesive 68 and may extend a distance “C” beyond the right edge 52 to a distance “D” towards the left edge 54. Further, the configuration of the second liner 78 may define a hinge 76 as described above. The window 70, as illustrated in FIGS. 5 and 6, may be formed in the foam 66 and the third adhesive 68 wherein the perimeter 71 of the window 70 may be defined by the foam 66.

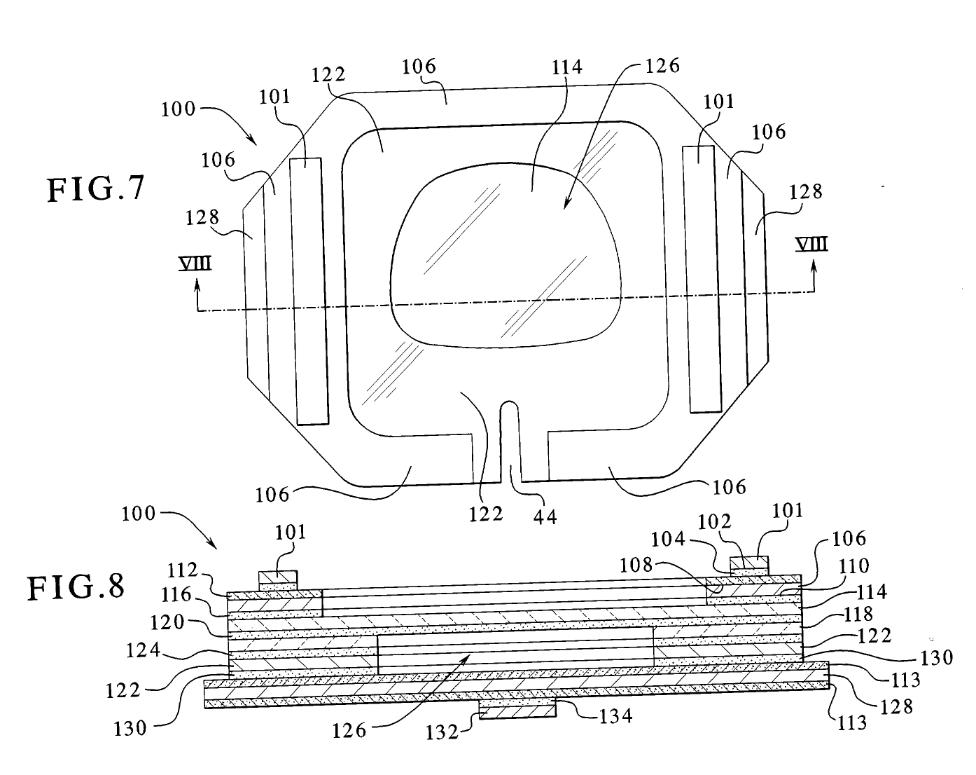

Referring now to FIGS. 7 and 8, in another embodiment of the present invention, a dressing 100 may have a strip 101 provided on a bottom side 102 with a first adhesive 104. Preferably, the strip 101 is constructed from non-woven polyester material. The non-woven polyester material may be flexible. A first liner 106 having a bottom surface 110 and a non-stick layer 112 on a top surface 108 of the first liner 106 may be attached to the bottom side 102 of the strip 101 by the first adhesive 104.

A first film 114 may be attached to the first liner 106 with an adhesive 116 on a side opposite from the strip 101. Preferably, the first film 114 is constructed from polyurethane and may be transparent. In an embodiment, the adhesive 116 that attaches the first film 114 with the first liner 106 may be a hot melt adhesive. Alternatively, the adhesive 116 may be a pressure sensitive adhesive. Preferably, the adhesive 116 may be an acyclic adhesive. A second film 118 may be attached to the first film 114 by a second adhesive 120. Preferably, the second film 118 is constructed from polyurethane and may be transparent. A layer 122 may be attached to the second film 118 on a side opposite the first film 114 by a third adhesive 124. Preferably, the layer 122 is constructed from a non-woven material that is flexible and soft to the touch to provide additional comfort to the patient.

A window 126 may be formed in the layer 122. A second liner 128 provided on both sides with a second non-stick layer 113 may be attached to the layer by a fourth adhesive 130. The second liner 128 may further cover the window 126. A label 132 may be attached to the second liner 128 on a side opposite from the layer 122 by another adhesive 134.

The label 132 may be used for, for example, documentation purposes. The label 132 may be removably attached to the second non-stick layer 113 on the second liner 128. More specifically, the second non-stick layer 113 provided on the second liner 128 may provide for easy removal of the label 132 from the dressing 100. For example, the information label 132 may be removed from the dressing 100 and may be applied to patient records, charts, or the like.

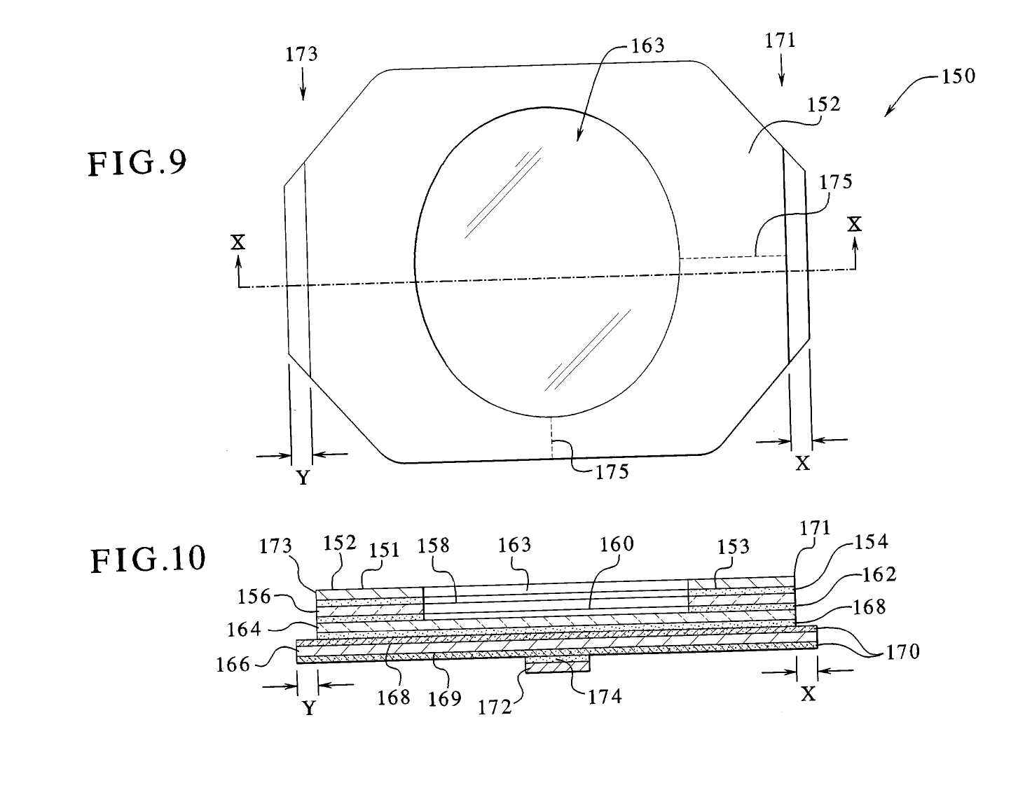

Referring now to FIGS. 9 and 10, in another embodiment of the present invention, a dressing 150 may have a first liner 152. An adhesive 154 may be provided on a bottom side 153 of the first liner 152. Preferably, the adhesive 154 may have a low adhesion property. A first film 156 having a top surface 158 and a bottom surface 160 may be attached to the adhesive 154 on the first liner 152. More specifically, the top surface 158 may be attached to the adhesive 154 on the bottom side 153 of the first liner 152. Preferably, the first film 156 may be constructed from polyethylene and may be transparent. The first film 156 may have a second adhesive 162 on the bottom surface 160. A second film 164 may be attached to the second adhesive 162 on the bottom surface 160 of the first film 156. Preferably, the second film 164 is constructed from polyurethane and may be transparent. A window 163 in the first liner 152, the adhesive 154, the first film 156 and the second adhesive 162 may allow for viewing of a wound and/or catheter insertion area through the second film 162.

A second liner 166 may be attached to the second film 164 by a third adhesive 168 on a side opposite from the first film 156. The second liner 166 may be constructed from paper or the like. Further, the second liner 166 may have a top 168 and a bottom 169. The top 168 of the second liner 166 may have a silicone layer 170. The bottom 169 of the second liner 166 may also have the silicone layer 170. A label 172 may be attached to the silicone layer 170 on the bottom 168 of the second liner 164 with a fourth adhesive 174. Preferably, the adhesive 154, the second adhesive 162, the third adhesive 168 and the fourth adhesive 174 may be acrylic based adhesives.

In an embodiment, the dressing 150 may not have the first film 156 with the second adhesive 162. In an embodiment, the second liner 166 may extend a distance “X” and a distance “Y” from a first edge 171 and a second edge 173 of the first liner 152, respectively. In another embodiment, the dressing 150 may have a slit 175. The slit 175 may allow for removal of the first liner 152 from the dressing 150.

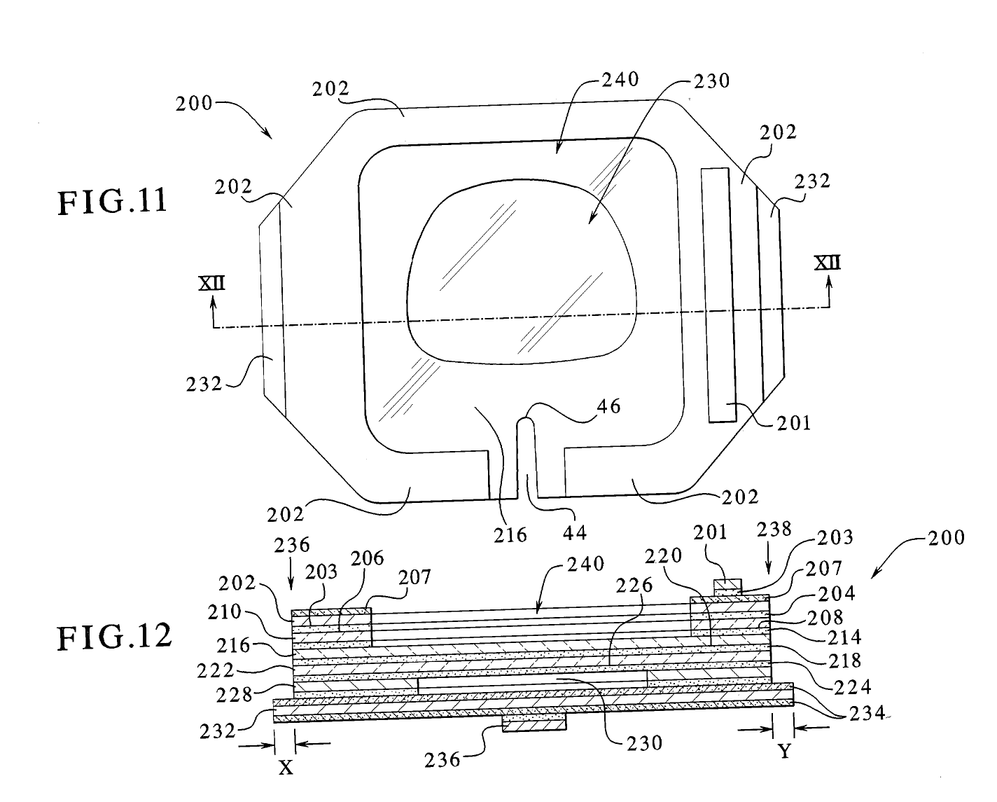

Referring now to FIGS. 11 and 12, in another embodiment of the present invention, a dressing 200 may have a first liner 202. A first adhesive 204 may be provided on a bottom side 203 of the first liner 202. In an embodiment, a label 201 may be attached to a top side 203 of the first liner 202 with an adhesive 205. The top side 203 of the first liner 202 may have a non-stick coating 207. A first film 210 having a top surface 206 and a bottom surface 208 may be attached to the first adhesive 204 on the first liner 202. More specifically, the top surface 206 of the first film 210 may be attached to the first adhesive 204 on the bottom side 203 of the first liner 202. Preferably, the first film 210 may be constructed from polyethylene and may be transparent.

The first film 210 may have a second adhesive 214 on the bottom surface 208. A second film 216 may be attached to the second adhesive 214 on the bottom surface 208 of the first film 210. Preferably, the second film 216 is constructed from polyurethane and may be transparent. The second film 216 may have a third adhesive 218 on a bottom side 220 of the second film 216. In an alternate embodiment, the dressing 200 may not have the first film 210 or the second adhesive 214. A third film 222 may be attached to the third adhesive 218 on the bottom side 220 of the second film 216. Preferably, the third film 222 is constructed from polyurethane and may be transparent. The third film 222 may have a fourth adhesive 224 on a side 226 opposite from the second film 216.

A layer 228 may be attached to the third film 222 by the fourth adhesive 224. Preferably, the layer 228 is constructed from a non-woven material that is flexible. The flexible non-woven material may provide comfort to the patient. A window 230 may be formed in the layer 228. A second liner 232 provided on both sides with a non-stick layer 234 may be attached to the layer 228 by a fifth adhesive 236. A second label 236 may be attached to the second liner 232 on a side opposite from the layer 228 by another adhesive 238. A top window 240 in the first liner 202 may allow for viewing of a wound and/or catheter insertion area through the first film 210, the second film 216, the third film 222 and the layer 228.

In an embodiment, the second liner 232 may extend a distance “X” and a distance “Y” from a first edge 236 and a second edge 238 of the first liner 202, respectively. In another embodiment, the dressing 200 may have a notch 44 having an end 46 to accommodate a cylindrical tube, such as, for example, a catheter. The notch 44 may simply be a slit or have the shape of a circle, square, or the like.

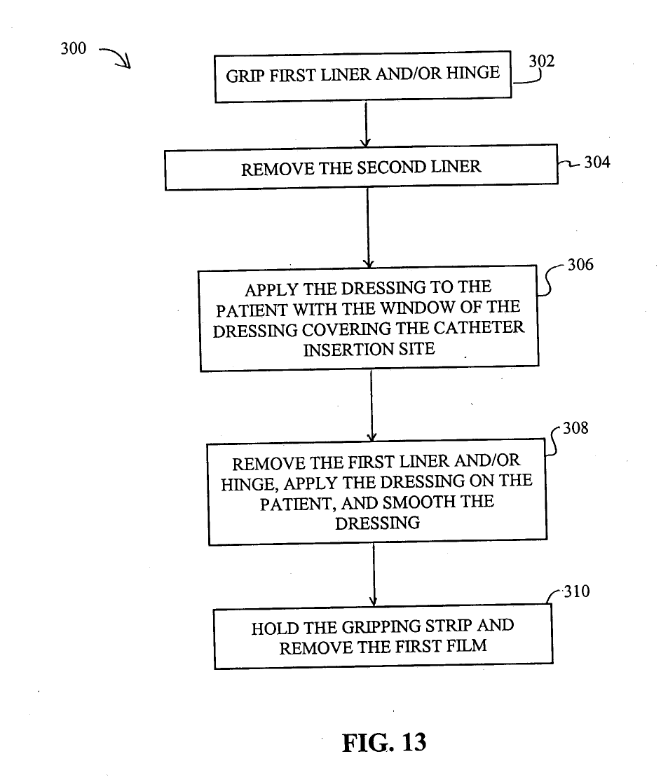

Referring to FIG. 13, a flowchart 300 showing a method for applying the dressing 10 of the present invention is generally illustrated. An individual, such as a medical professional, patient, or the like, may grip the first liner 28 and/or the hinge 42 formed by the first liner 28 as shown at step 302. The individual may remove the second liner 36 from the dressing 10 as shown at step 304. The individual may then apply the dressing 10 to the patient with the window 34 of the dressing 10 covering the catheter insertion site as shown at step 306. The individual may then remove the first liner 28 and/or the hinge 42, apply the dressing 10 on, for example a patient, and smooth the dressing 10 as shown at step 308. The individual may then hold the gripping strip 20 and remove the first film 12 as shown at step 310.

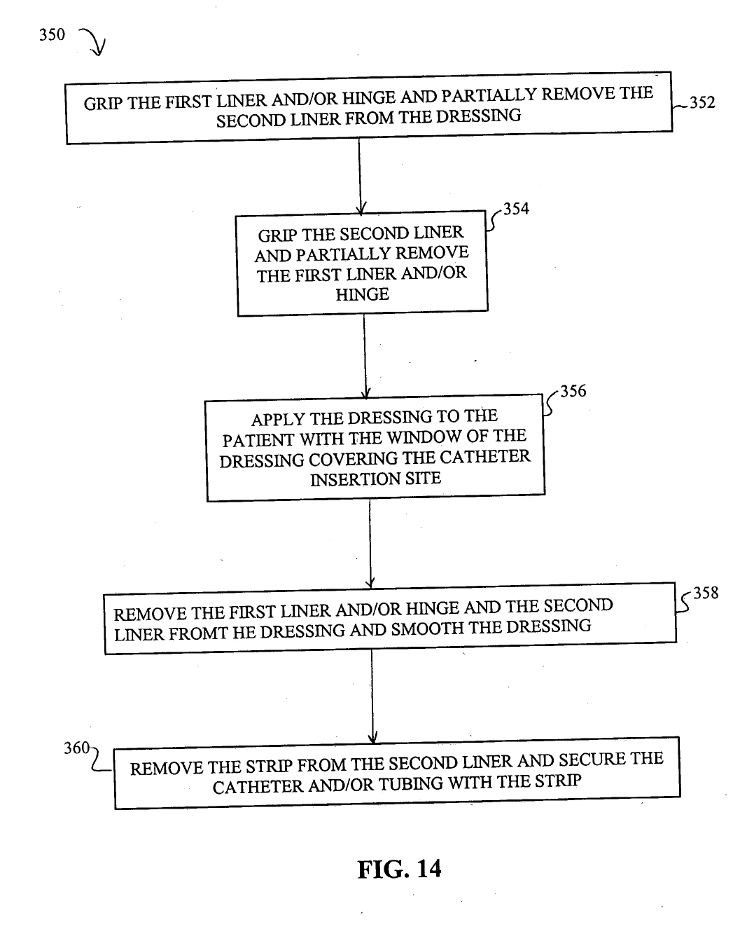

Referring to FIG. 14, a flowchart 350 showing a method for applying the dressing 50 of the present invention is generally illustrated. The individual may grip the first liner 72 and/or the hinge 76 and may partially remove the second liner 78 from the dressing 50 as shown at step 352. The individual may then grip the second liner 78 and partially remove the first liner 72 and/or the hinge 76 from the dressing 50 as shown at step 354. The individual may then apply the dressing 10 to the patient with the window 70 of the dressing 50 covering the catheter insertion site as shown at step 356. The individual may then remove the first liner 28 and/or the hinge 42 and the second liner 78 from the dressing 50 and smooth the dressing 50 as shown at step 358. The individual may then remove the strip 82 from the second liner 78 and may secure the catheter and/or tubing with the strip 82 as shown at step 360.

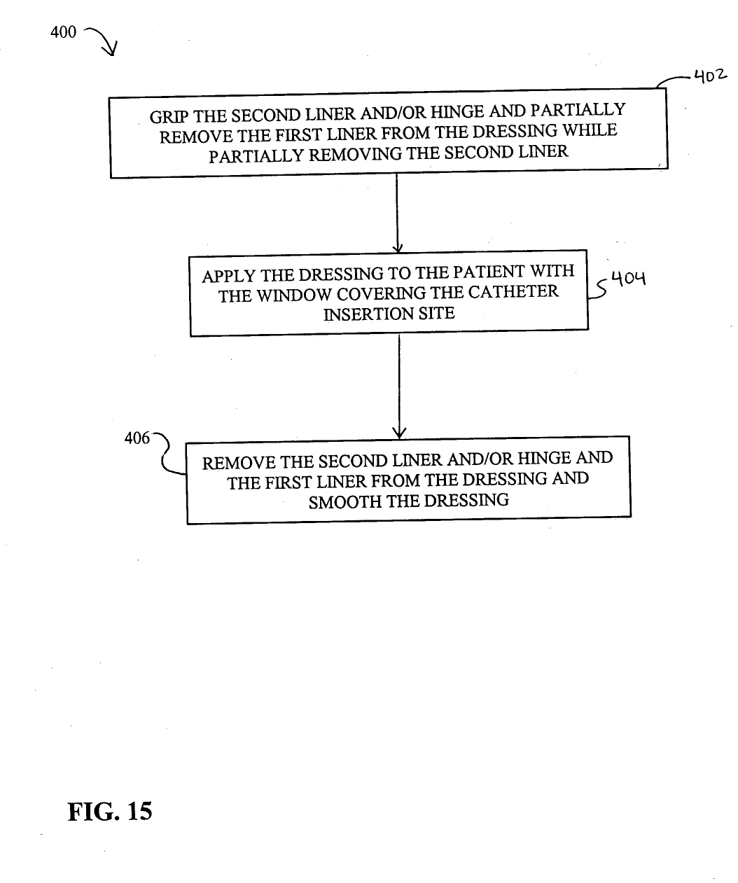

Referring to FIG. 15, a flowchart 400 showing a method for applying the dressing 90 of the present invention is generally illustrated. The individual may grip the second liner 78 and/or the hinge 76 and may partially remove the first liner 78 from the dressing 90 while partially removing the second liner 78 as shown at step 402. The individual may then apply the dressing 90 to the patient with the window 70 of the dressing 90 covering the catheter insertion site as shown at step 404. The individual may then remove the second liner 78 and/or the hinge 76 and the first liner 72 from the dressing 90 and smooth the dressing 90 as shown at step 406.

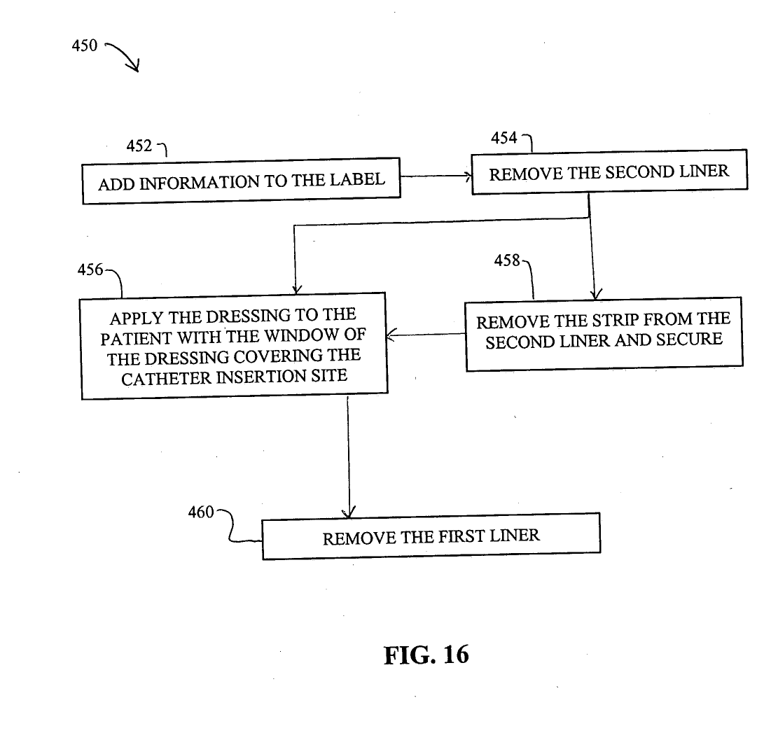

Referring to FIG. 16, a flowchart 450 showing a method for applying the dressing 100 of the present invention is generally illustrated. The individual may add information to the label 132 of the dressing 100 as shown at step 452. Alternatively, the individual may skip step 452 and may remove the second liner 128 as shown at step 454. After removal of the second liner 128, the individual may then apply the dressing 100 to the patient with the window 126 of the dressing 100 covering the catheter insertion site as shown at step 456. The individual may then remove the strip 101 from the second liner 128 and may secure the catheter and/or tubing with the strip 101 as shown at step 458. The individual may skip step 458 and remove the first liner 106 from the dressing 90 as shown at step 460.

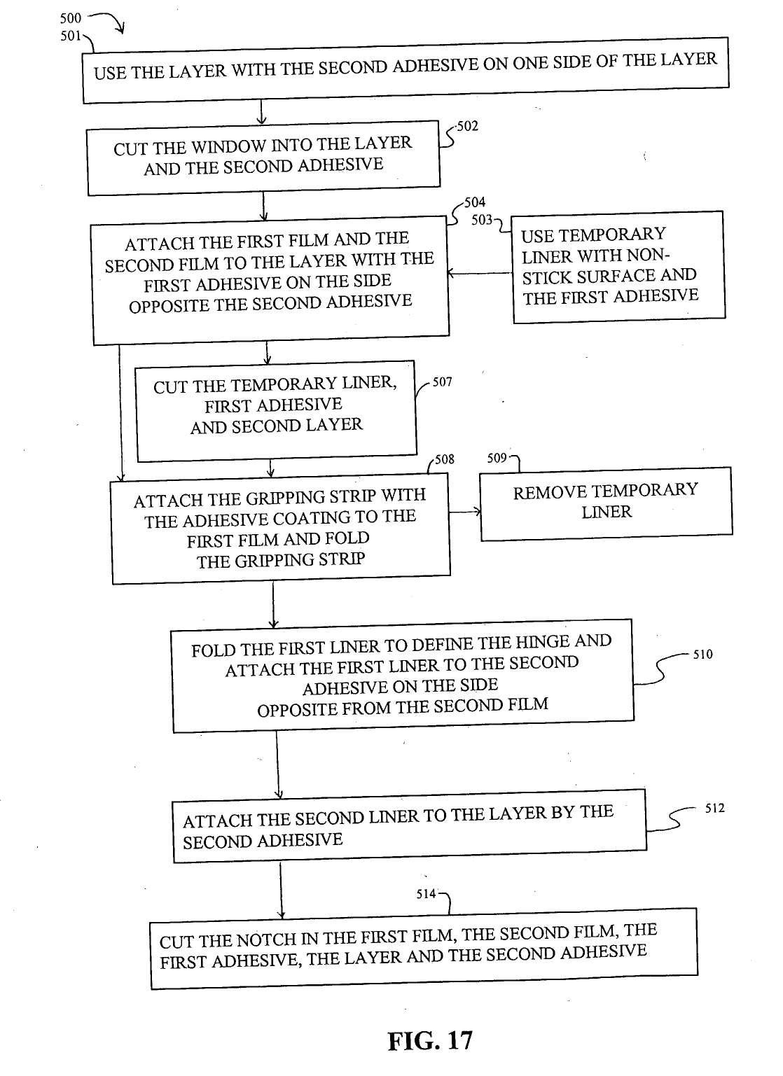

A flowchart 500 illustrating an embodiment of a method to manufacture the dressing 10 of the present invention is generally illustrated in FIG. 17. The layer 24 with the second adhesive 32 on one side of the layer 24 may be used as shown at step 501. The window 34 may be cut into the layer 24 and the second adhesive 32 as shown at step 502. The first film 12 and the second film 18 may be attached to the layer 24 with the first adhesive 26 on the side opposite the second adhesive 32 as shown at step 504. The gripping strip 20 with the adhesive coating 23 may be attached to the first film 12 and may be folded as shown at step 508.

The first liner 28 may be folded to define the hinge 42 and may be attached to the second adhesive 32 on the side opposite from the second film 18 as shown at step 510. The second liner 36 with the second silicone coating 38 may be attached to the layer 24 by the second adhesive 32 as shown at step 512. The notch 44 may be provided in the dressing 10 by cutting, for example, a circle, a slit, or the like through the first film 12, the second film 18, the first adhesive 26, the layer 24, and the second adhesive 32 as shown in step 514.

In an embodiment, a temporary liner having a non-stick coating on a top and bottom surface and the first adhesive 26 on the non-stick coating on the top surface may be used as shown at step 503. The first adhesive 26 may preferably be an acrylic based adhesive. The first adhesive 26 on the temporary liner may be cured prior to use. After curing the temporary liner, the second film 18 may be applied to the top surface of the temporary liner by the first adhesive 26 as shown at step 504. The second film 18, the first adhesive 26 and the temporary liner may be cut to the width of the dressing 10 as shown at step 507. As described above, the first film 12 may be attached to the second film 18. Further, the gripping strip 20 with the adhesive coating 23 may be attached to the first film 12 and may be folded as shown at step 508. The temporary liner may be removed as shown at step 509 and the layer 24 may be attached to the second film 18 with the first adhesive 26 on the side opposite the second adhesive 32 as shown at step 504.

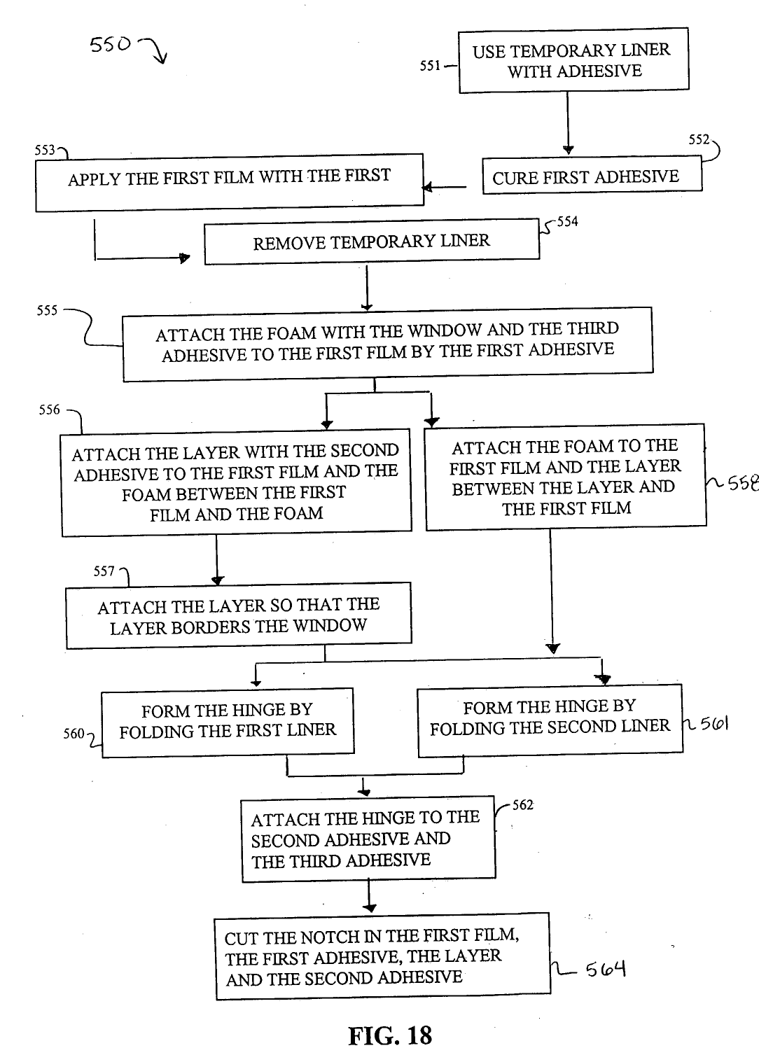

A flowchart 550 illustrating another embodiment of a method to manufacture the dressing of the present invention is generally illustrated in FIG. 18. A temporary liner (not shown) having a top surface and a bottom surface may be used as shown at step 551. The temporary liner may have a non-stick coating on the top and bottom surface and may further have the first adhesive 60 on the top surface of the temporary liner. The first adhesive 60 may preferably be an acrylic based adhesive. The temporary liner with the first adhesive 60 may be cured as shown at step 552. After curing the temporary liner, the first film 51 may be applied to the top surface of the temporary liner by the first adhesive 60 as shown at step 553. The temporary liner may be removed as shown at step 554. The foam 66 with the window 70 and the third adhesive 68 may be attached to the first film 51 by the first adhesive 60 as shown at step 555. The layer 62 with the second adhesive 64 may also be attached to the first film 51 and the foam 66 wherein a portion of the layer 62 is between the first film 51 and the foam 66 as shown at step 556. The layer 62 may border the window 70 as shown at step 557. Alternatively, the foam 66 may be attached to the first film 51 and the layer 62 wherein a portion of the foam 66 is between the layer 62 and the first film 51 as shown at step 558.

The hinge 76 may be formed by folding the first liner 72 as shown at step 560. The hinge 76 may be attached to both the second adhesive 64 and the third adhesive 68 as shown at step 561. Alternatively, the hinge 76 may be formed by folding the second liner 78 and attaching the hinge 76 to both the second adhesive 64 and the third adhesive 68 as shown at step 562. A notch 44 may be provided in the dressing 50 by cutting through the first film 51, the first adhesive 60, the layer 62, and the second adhesive 64 for a catheter as shown in step 564.

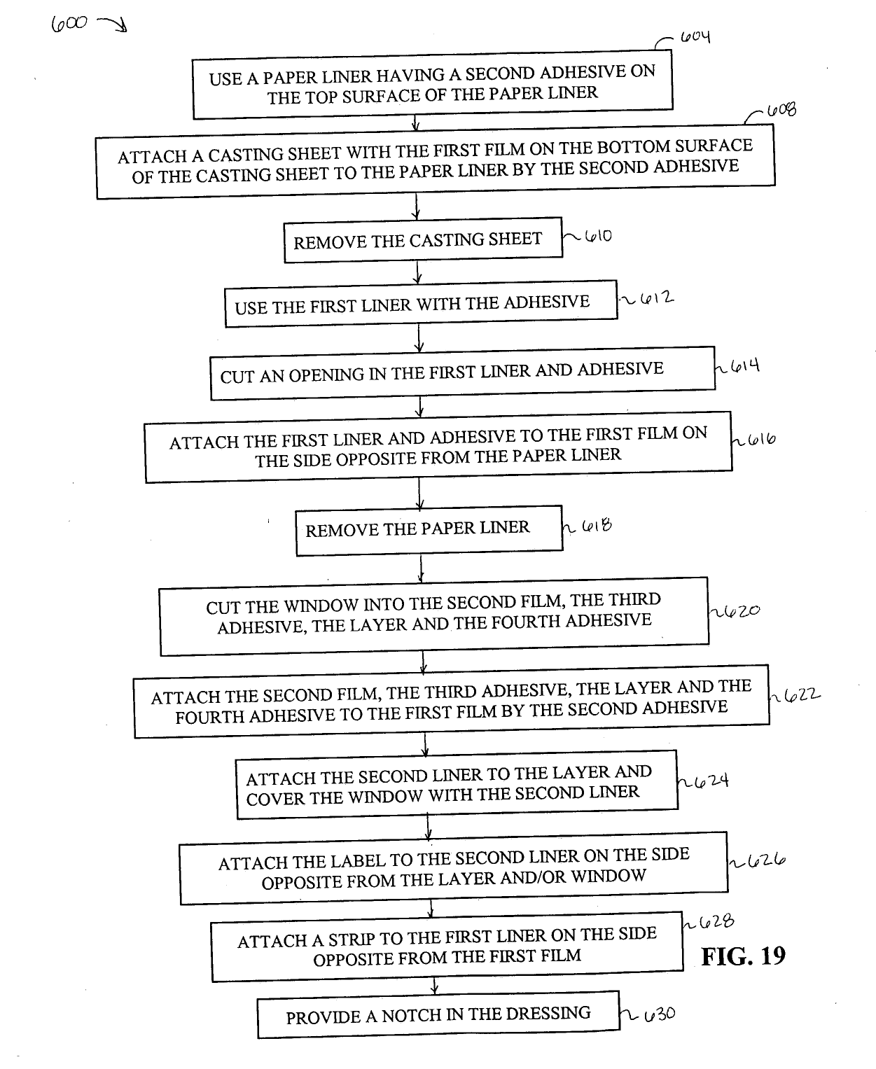

A flowchart 600 illustrating a further embodiment of a method to manufacture the dressing 100 of the present invention is generally illustrated in FIG. 19. A paper liner (not shown) having a top surface and a bottom surface and a non-stick coating on the top surface and may further have the second adhesive 120 on the top surface of the paper liner may be used as shown at step 604. The second adhesive 120 may preferably be an acrylic based adhesive. The paper liner with the second adhesive 120 may be cured prior to use. A casting sheet (not shown) with a bottom surface and having the first film 114 on the bottom surface may be attached to the paper liner by the second adhesive 120 as shown at step 608. The casting sheet may be removed as shown at step 610. The first liner 106 with the is adhesive 116 may be used as shown at step 612. An opening may be cut into the first liner 106 and adhesive 116 as shown at step 614. The first liner 106 and adhesive 116 may be attached to first film 114 on the side opposite from the paper liner as shown at step 616. The paper liner may be removed as shown at step 618. The window 126 may be cut into the second film 118, the third adhesive 124, the layer 122, and the fourth adhesive 130 as shown at step 620. The second film 118, the third adhesive 124, the layer 122, and the fourth adhesive 130 may be attached to the first film 114 by the second adhesive 120 as shown at step 622. In an alternative embodiment, the adhesive 116 may be hot-melted and applied to the first liner 106 prior to attaching the first film 114. Further, in another embodiment, the second film 118 may be laminated to the first film 114.

The second liner 128 may be attached to the layer 122 and may further cover the window 126 as shown at step 624. The label 132 may be attached to the second liner 128 on the side opposite from the layer 122 and/or the window 126 as shown at step 626. The strip 101 having the first adhesive 104 may be attached to the first liner 106 on the side opposite from the first film 114 as shown at step 628. A notch 44 may be provided in the dressing 100 by cutting through the first film 114, the second film 118, the layer 122, the second liner 128, and intervening adhesives as shown at step 630.

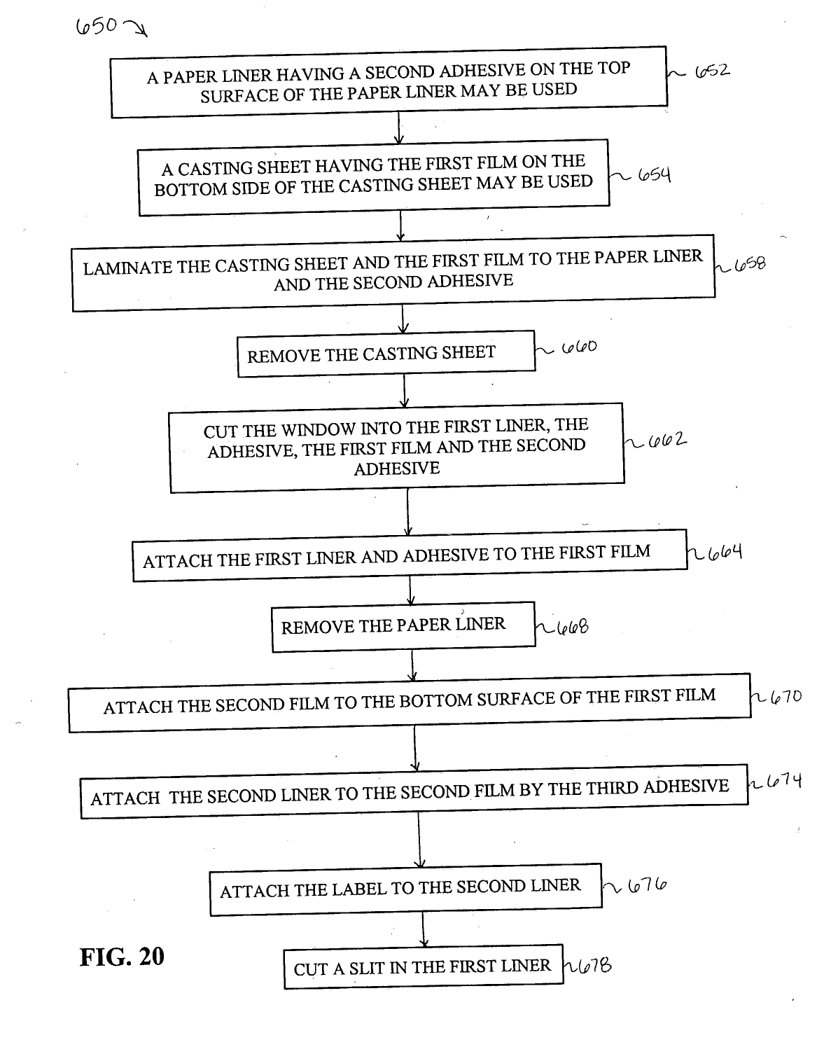

A flowchart 650 illustrating another embodiment of a method to manufacture the dressing 150 of the present invention is generally illustrated in FIG. 20. A paper liner (not shown) having a non-stick coating on the top and bottom surface and having the second adhesive 162 on the top surface of the paper liner may be used as shown at step 652. The second adhesive 162 may preferably be an acrylic based adhesive and may be cured prior to use. A casting sheet (not shown) having the first film 156 on a bottom side of the casting sheet may be used as shown at step 654. The casting sheet and first film 156 may be laminated to the paper liner and second adhesive 162 as shown at step 658. The casting sheet may be removed as shown at step 660. The window 163 may be cut into the first liner 152, the adhesive 154, the first film 156 and the second adhesive 162 as shown at step 662. The first liner 152 and adhesive 154 may be attached to the first film 156 on a side opposite from the paper liner as shown at step 664. The paper liner may be removed as shown at step 668.

The second film 164 may be attached to the bottom surface 160 of the first film 156 as shown at step 670. The second liner 166 having silicone layer 170 on the top 168 and the bottom 169 of the second liner 166 may be attached to the second film 164 by the third adhesive 168 opposite from the first film 156 as shown at step 674. The label 172 may be attached to the bottom 168 of the second liner 164 as shown at step 676. Further, the first liner 152 may be provided with the slit by cutting the first liner 152 as shown at step 678.

In another embodiment of a method to manufacture the dressing of the present invention, a paper liner (not shown) having an acrylic adhesive may be used. The paper liner with the acrylic adhesive may be provided in a pre-manufactured roll. A roll of film may be applied to the liner such that the acrylic based adhesive secures the film to the liner. The film and the liner may then be pressed together. The film and the paper liner may be cut to match the width of the dressing. The paper liner may be removed and various layers may be attached to the film.

It should be appreciated that the dressing may be any number of shapes and/or sizes including rectangles, circles, triangles, and the like. Further, the dressing may be any number of different colors, such as, for example, white or blue. The colors of the dressing may indicate the size of the dressing. The different sized dressings may be used for different sized wounds and/or different sized catheters. Still further, instructions may be printed on the liner. Instructions may include, for example, to remove the liner first when applying the dressing. Alternatively, the instructions may be printed as part of a pattern that may cover a portion of the liner or all of the liner.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

I claim:

1. A dressing comprising:
a first film having right edge, a left edge, a top side and a bottom side;
a first layer having a top surface and a bottom surface wherein the top surface of the first layer is attached to the bottom side of the first film;
a first adhesive on the bottom surface of the first layer;
a second layer having a top face, a bottom face and a second adhesive on the bottom face wherein the top face of the second layer is attached to the bottom surface of the first layer by the first adhesive; and
a liner having a first side and a second side and a non-adhesive coating on the first side wherein the liner is removably attached to the bottom face of the second layer.

2. The dressing of claim 1 further comprising:
a notch in the first film.

3. The dressing of claim 1 further comprising:
a strip attached to the top side of the first film.

4. The dressing of claim 1 further comprising:
a window in the second layer.

5. The dressing of claim 1 further comprising:
a hinge secured to the second layer.

6. The dressing of claim 1 further comprising:
a third adhesive securing the first layer to the bottom side of the first film.

7. The dressing of claim 1 further comprising:
a second non-adhesive layer on the second side of the liner.

8. The dressing of claim 1 further comprising:
a second liner having a top and a bottom wherein the bottom of the second liner is secured to the top side of the first film.

9. The dressing of claim 1 further comprising:
a second liner having a strip wherein the second liner is secured to the top side of the first film.

10. The dressing of claim 1 further comprising:
a label removably attached to the second side of the liner.

11. The dressing of claim 1 wherein the first layer is made from a foam.

12. The dressing of claim 1 wherein the first layer is made from a film.

13. The dressing of claim 1 wherein the first film is made from a polyethylene material.

14. The dressing of claim 1 wherein the first film is made from a polyurethane material.

15. The dressing of claim 1 wherein the second layer is made from a foam.

16. The dressing of claim 1 wherein the first layer is made from a non-woven polyester material.

17. The dressing of claim 1 wherein the first adhesive includes acrylic.

18. A dressing comprising:
a frame having a top and a bottom wherein the bottom of the frame has an adhesive;
a first film having a top side and a bottom side wherein the top side of the first film is attached to the bottom of the frame;
a second adhesive on the bottom side of the first film;
a second film having a top surface and a bottom surface wherein the top surface of the second film is attached to the bottom side of the first film by the second adhesive; and
a third adhesive on the bottom surface of the second film.

19. The dressing of claim 18 further comprising:
a second liner secured to the bottom surface of the second film by the third adhesive.

20. The dressing of claim 19 further comprising:
a non-adhesive layer on the second liner.

21. The dressing of claim 18 further comprising:
a third film attached to the bottom surface of the second film by the third adhesive.

22. The dressing of claim 18 wherein the frame has a shape and the first film has a shape wherein the shape of the first film is substantially the same as the shape of the frame.

23. A method for applying a dressing to a catheter insertion site on a patient, the method comprising the steps of:
providing a frame having a top and a bottom wherein the bottom of the frame has an adhesive;
providing a first film having a bottom side;
attaching the first film to the bottom of the frame with the adhesive;
providing a first layer having a top surface and a bottom surface;
attaching the top surface of the first layer to the bottom side of the first film;
providing a second layer having a top face and a bottom face wherein the top face of the second layer is on the bottom surface of the first layer; and
exposing the bottom face of the second layer.

24. The method of claim 23 further comprising the step of:
placing the second layer on the patient.

25. The method of claim 23 further comprising the step of:
securing the dressing on the patient with an adhesive strip.

26. A method for manufacture of a dressing, the method comprising the steps of:
providing a temporary liner with an adhesive;
attaching a first film to the temporary liner with the adhesive;
removing the temporary liner;
attaching a first layer to the first film;
providing a second layer having a top face, a bottom face and having a second adhesive on the bottom face;
attaching the first film to the top face of the second layer with the adhesive;
securing a liner to the second layer; and
attaching a second liner to the first layer.

27. The method of claim 26 further comprising the step of:
providing a casting sheet having the first film and removing the casting sheet after attaching the first film to the temporary liner.

28. The method of claim 26 further comprising the step of:
curing the adhesive on the temporary liner.

29. The method of claim 26 further comprising the step of:
forming a notch in the dressing.

30. The method of claim 26 further comprising the step of:
attaching the second liner to the bottom face of the second layer with the second adhesive.

31. The method of claim 26 further comprising the step of:
attaching a removable label to the liner.

32. The method of claim 26 wherein the first layer is a polyurethane film.

33. The method of claim 26 wherein the first film is a polyethylene film.

34. The method of claim 26 wherein the second layer is a polyethylene foam.

35. The method of claim 26 further comprising the step of:
securing a strip to the liner.

36. The method of claim 26 further comprising the step of:
forming a window in the second layer.

37. The dressing of claim 26 wherein the adhesive includes acrylic.
